(19) United States
(12) Patent Application Publication
(10) Pub. No.: US 2019/0267126 A1
(43) Pub. Date: Aug. 29, 2019

(54) SUPPORT SYSTEM AND METHOD FOR SURGICAL INTERVENTION

(71) Applicants: Kristoff CORTEN, Langdorp (BE); INCORDEV, Herselt (BE)

(72) Inventor: Kristoff CORTEN, Langdorp (BE)

(21) Appl. No.: 16/344,247

(22) PCT Filed: Oct. 24, 2017
(86) PCT No.: PCT/IB2017/056599

§ 371 Date: Apr. 23, 2019

(30) Foreign Application Priority Data
Oct. 24, 2016 (BE) 2016/5797

(51) Int. Cl. G16H 20/40; G16H 40/20

(52) U.S. Cl. CPC G16H 20/40; G16H 40/20

(57) ABSTRACT

A system for supporting a surgical intervention to be carried out on a patient comprises at least one means of identification that can be fitted to the patient, medical instruments with means of identification for surgical intervention, and a computer system. The medical instruments comprise at least a surgical drape. The computer system comprises a list of the various medical instruments necessary for the surgical intervention to be carried out. The computer system has been amended to identify the means of identification electronically, and to carry out a program that allows the surgical intervention only when these means of identification have been identified which correspond with the medical instruments stated on the list.

### SCOPE OF THE INVENTION

This invention generally relates to systems and methods for supporting a surgical or other invasive intervention (such as e.g. anaesthesia methods). More specifically, it relates to systems and methods contributing to the planned and desired medical intervention being correctly prepared and started on the patient in question. The system implements a safety system in existing and new medical products, or products relating to medical interventions, so that the material provides feedback and confirmation to the medical and/or paramedical team that the intervention anticipated was prepared correctly on the patient in question up to the start of the intervention.

### BACKGROUND OF THE INVENTION

In order to prevent errors and side effects as a result of medical interventions, the World Health Organisation has developed a “Surgical Safety Checklist”. ‘Intervention’ means any medical act or procedure undertaken that has an invasive character. Not restrictively, this can include acts such as carrying out a puncture, taking a blood sample, placing a drip, various anaesthesia methods and surgical procedures.

The “Safety Checklist” consists of a number of questions relating to the ‘procedure’ that need to be ticked by the various parties involved such as the nurse(s) and the physicians (e.g. the anaesthetist and the surgeon). A number of questions and parameters must be ticked first during the preparation of the procedure and so before the medical act takes place. This checklist is intended to minimise the chance that the incorrect procedure is prepared and started on the patient in question. In other words, the checklist is intended to verify that the correct procedure was prepared and initialised on the correct patient. In order to achieve this, a number of questions need to be ticked during the entire preparatory process. Furthermore, a number of questions and parameters need to be ticked before the patient leaves the operating room.

But even with the use of such a list, there is still the chance of human error. This is due to the fact that during the preparatory process, there are many different parties involved, so mistakes can easily occur:
(1) Patient intervention planning: The incorrect intervention can be planned (e.g. during the consultation) e.g. by the physician or the secretary and this without the patient having confirmed the intervention
(2) Material: In the preparation of the operation, the incorrect materials can be ordered by the person in charge of material who must check that specific material for an intervention is present at the hospital
(3) Material: The incorrect material is made available in the pre-operation logistics area for the intervention, e.g. by the person in charge of logistics
(4) Material: The incorrect material is brought into the operating room e.g. by the nurse
(5) Patient admittance to hospital: The incorrect intervention is allocated to the patient during the admittance process in the hospital
(6) Patient transfer/admittance to operating room: The incorrect patient is brought to the operating area e.g. by the person in charge of logistics
(7) Patient anaesthesia: The incorrect anaesthesia method and site and preparation is carried out by the anaesthesiology team
(8) Operation preparation: The incorrect material is prepared by the surgical team
(9) Operation preparation: The surgeon is intending to start the incorrect intervention

The result is that the incorrect intervention can happen to the incorrect patient, using the incorrect material but this can also lead to delay in the surgical intervention, for example because some materials have not been made available or some incorrect materials have been anticipated or the necessary materials are not even present. The above-mentioned steps of the entire preparatory process are currently not included in the ‘Safety Checklist’. Furthermore, confirmation is not always asked from all the parties involved that the correct execution of the relevant step is being met.

In addition to such a ‘safety checklist’ to prevent errors and secondary effects as a result of medical interventions, there is still room for systems and methods for supporting the correct preparation and planning of a medical procedure. Moreover, the patient must not sign a consent at the start of the Safety Checklist. In other words: There is no double confirmation that the patient agrees with the intervention planned.

Finally, the current ‘Safety Checklist’ is not only incomplete in comprising the entire process, but the Safety Checklist is also very time-consuming from an administrative point of view.

### SUMMARY OF THE INVENTION

It is an aim of embodiments of the present invention to anticipate methods and systems that help to have the desired ‘procedure’ run smoothly, but also to cancel out or at least minimise the chance that the wrong procedure was prepared for the patient in question.

A safety system that asks for confirmation from all the parties involved and that makes a ‘closed loop’ from the planning and confirmation by the patient to the final step (namely the initiation of the intervention), and the materials used forming an integral part of the safety system, will increase the efficiency and the accuracy of the Safety List. As the materials anticipated are also included in the Safety process, one has a unique additional check that the correct intervention was prepared and initiated on the patient in question. This patent application provides a final, ‘reactive’ step, the material anticipated providing pertinent feedback to the medical team that the material has or has not been anticipated to carry out the planned intervention. The aforementioned aim is achieved by an appliance, device and/or method according to the present invention.

The present invention relates to a system for supporting a surgical intervention to be carried out on a patient, the system comprising:
at least one means of identification, that can be fitted to the patient,
medical instruments provided with means of identification for a surgical intervention, and a computer system,
the computer system comprising a list of the various medical instruments necessary for the surgical intervention to be carried out,
the computer system having been amended to identify the medical instruments using means of identification in an electronic or other manner,
and the computer system having been programmed to execute a program that allows the surgical intervention only when these means of identification have been identified which correspond with the medical instruments stated on the list. The list can be part of the pre-programmed procedure that was confirmed by the patient. The medical instruments comprise at least a surgical drape, at least one means of identification having been fitted to the surgical drape positioned such that the final means of identification can be identified only when the surgical drape has been unfolded. Several means of identification can be used during the preparatory process but the final means of identification for the start of the intervention closes the ‘closed loop’ and gives the final signal regarding whether or not to start the correct intervention as was confirmed by the physician at the planning and the one confirmed by the patient on the (informed) consent form, in whichever form this informed consent form was used. The computer system has been amended to identify the means of identification in an electronic manner and the program has been amended to derive a surgical intervention from at least one means of identification on the surgical drape and to allow a surgical intervention to happen only when the derived surgical intervention corresponds with the surgical intervention to be carried out that was confirmed by all the parties involved, including the attending team of physicians and nurses but by the patient too.

It is an advantage of embodiments of the present invention that the correct medical instruments have been collected to be able to carry out the surgical intervention, that must be carried out on the patient. It is an advantage that the checking of this happens by computerisation. The means of identification, for example labels, are identified in an electronic manner and the verification that these means of identification correspond with the planned intervention and the medical instruments stated on the list is done using a program that is executed on a computer system. It is an advantage of embodiments of the present invention that it is not possible that an incorrect medical instrument is used for a certain surgical intervention. The advantage of the embodiments is that a ‘closed loop’ is created by the attending team at the planning, the patient at the confirmation (on the consent form) and by the means of identification on the materials to be used, the final means of identification possibly being made visible and giving feedback as the final step only once the entire preparatory process has been completed. As such, the loop is finally closed by the final means of identification and incorrect preparations of procedures are made impossible.

The program can furthermore demand that at least one confirmation about the surgical intervention to be carried out is given by a person before the program allows the operation.

It is an advantage of embodiments of the present invention that the surgical intervention to be carried out must be confirmed at least once by 3 different parties: the attending team (i.e. physician, nurse, secretary, logistics worker), the patient themselves and the final means of identification. In this manner, the loop is closed by all parties involved and it is prevented that the incorrect surgical intervention is carried out.

The program can therefore require that a confirmation about the surgical intervention to be carried out is given by the patient, the person in charge of logistics and/or the medical and/or paramedical team before the system allows the operation to happen.

Also, a patient's performance level as captured by the computer program can be a confirmation parameters too.

It is an advantage of embodiments of the present invention that an additional verification of the surgical intervention is done by the person in charge of logistics. This verification can for example happen after the person in charge of logistics has made the required medical instruments available.

The system can also comprise a means of identification that can be fitted to the patient, this means of identification allowing the patient to be identified.

It is an advantage of embodiments of the present invention that a means of identification on the patient allows the patient to be identified in the computer system. By this computerisation, the chance of an incorrect patient selection in the computer system is reduced. Furthermore, this can be further reduced by asking a confirmation from the patient. In embodiments of the present invention, the program has been amended to determine, based on the patient identified, which surgical intervention was planned, prepared and will be initiated. Also, in embodiments of the present invention, the surgical intervention can be reconfirmed again by the patient.

It is an advantage of embodiments of the present invention that by identifying a means of identification, for example label, on the surgical drape, it is possible to associate this means of identification with a surgical intervention. By identifying the means of identification on the surgical drape using the computer system, it is possible to verify an additional time that the surgical intervention corresponding with the means of identification corresponds with the surgical intervention to be carried out. It is furthermore an advantage that this verification can be done by computerisation.

At least one means of identification, for example label or (programmed) sensor, can be placed on the surgical drape such that when the surgical drape is placed correctly on the patient, the identification of the label corresponds with the site of a surgical intervention that needs to be carried out at the site of the label.

It is an advantage of embodiments of the present invention that by identifying a means of identification on the surgical drape, it is possible to associate this means of identification with a surgical intervention in this site. By identifying the means of identification on the surgical drape using the computer system, it is possible to verify an additional time that the surgical intervention corresponding with the means of identification which corresponds with the surgical intervention or other invasive procedure to be carried out.

The surgical drape can comprise protection foils and the means of identification can be released after one or several protection foils have been removed or the sensor can give a signal and communicate with the computer program only when the medical team activates the sensor.

It is an advantage of embodiments of the present invention that the means of identification, corresponding with the surgical intervention to be carried out, is released only after one or several protection foils have been removed. This system can verify that the correct protection foils have been reduced.

The computer program can furthermore have been programmed to register a patient outcome for the intervention. This can be a confirmation parameter for whether or not the computer system approves the operation. It is an advantage of the embodiments that a full integration is possible within the computer program between the pre and post operation status and follow-up of the patient on the one hand and the safety and correctness of the preparation of the intervention to be carried out on the other hand. After all, the computer program allows that the ‘closed loop’ of procedure safety as described above is built-in with a full platform of patient monitoring and steering of before and after the intervention. In addition to the confirmation of the consent form for the intervention, the patient also gives consent for a full and integral pre and post operation tracking system to be used, in which questionnaires regarding the performance, instructions and instruction videos regarding the rehabilitation process are used and made available. It is an advantage of the embodiments that the elements of the computer program and the means of identification make it possible for a database to be constructed with data such as safety parameters, surgical parameters, performance modalities and rehabilitation parameters of the patient population making use of the computer program and the embodiments. Finally, also the electronic means of identification, which the patient fitted him or herself in the form of performance scoring lists, can be used to allow an intervention to happen or not. This could prevent that patients who are insufficiently affected, or whose performance is insufficiently poor, would have the intervention. This creates an extra-closed loop, the intervention not being based on a planning only, but also on a performance level the patient who has also confirmed the intervention within the program.

The present invention also relates to a method for supporting a surgical intervention, this method comprising:
an identification step for identifying the patient and the intervention and one or several medical instruments in an electronic manner using a means of identification on the medical instruments, the medical instruments comprising at least a surgical drape, at least one means of identification having been fitted to the surgical drape and positioned such that the means of identification can be identified only when the surgical drape has been unfolded and is ready for use,
a verification step for checking a list of medical instruments required for the surgical intervention with the identified medical instruments, a surgical intervention being derived from at least one means of identification on the surgical drape and the operation being allowed to happen only when these labels have been identified which correspond with the medical instruments stated on the list and when the derived surgical intervention corresponds with the surgical intervention to be carried out.

The method can furthermore comprise a confirmation step in which the surgical intervention is confirmed by a person and by the patient.

During the identification step, the patient can also be identified using a means of identification on the patient.

During the identification step, a means of identification can be identified on a surgical drape and the identification of this means of identification is associated with a specific surgical intervention.

During the verification step, it can be verified that the surgical intervention to be carried out corresponds with the surgical intervention or the identified means of identification on the surgical drape.

Before or during the verification step, protection foils can be taken away from the surgical drape so that the means of identification is released or the sensor is activated. Alternatives for the protection foils can be used too. The means of identification must be activated in whichever manner and must therefore be able to communicate/interact with the computer system before initiating the intervention. Releasing the means of identification is regarded as an activation step.

The present invention relates furthermore to a medical instrument, a means of identification having been fitted that can be identified by a system such as described above.

The medical instrument is a surgical drape that comprises a means of identification that can be identified only after the surgical drape has been unfolded.

Specific and preferable aspects of the invention have been included in the attached independent and dependent claims. Features of the dependent claims can be combined with features of the independent claims and with features of other dependent claims such as indicated and not only as expressly brought forward in the claims.

These and other aspects of the invention will be apparent from and be clarified by reference to the embodiment(s) described below.

### SHORT DESCRIPTION OF THE DRAWINGS

FIG.1 is a schematic representation of a system for supporting a surgical intervention on a patient corresponding with embodiments of the present invention.

FIG. 2 shows a possible succession of the various steps of a method corresponding with embodiments of the present invention.

The drawings are only schematic and not restrictive. It is possible that the dimensions of some components have been exaggerated and have not been represented to scale in the drawings for illustrative purposes.

Reference numbers used in the claims must not be interpreted to restrict the scope of protection. In the various drawings, the same reference numbers refer to the same or similar elements.

### DETAILED DESCRIPTION OF ILLUSTRATIVE EMBODIMENTS

The present invention will be described in respect of special embodiments and with reference to certain drawings, however the invention will not be restricted to this but will be restricted only by the claims. The drawings described are only schematic and not restrictive. In the drawings, the dimensions of some elements may have been enlarged and not have been drawn to scale for illustrative purposes. The dimensions and the relative dimensions sometimes do not correspond with the up-to-date practical embodiment of the invention.

Furthermore, the terms first, second, third and the like in the description and in the claims are used to distinguish similar elements and are not necessarily used for describing an order, nor in time, nor in space, nor in ranking nor in any other manner. It should be understood that the terms used in this way are interchangeable in appropriate circumstances and that the embodiments of the invention described herein are suitable to work in a different order than described or indicated in here.

It should be noted that the term “comprises”, as used in the claims, should not be interpreted as being restricted to the items described thereafter; this term does not exclude any other elements or steps. It may be interpreted as specifying the presence of the features, values, steps or components indicated which are referred to, but does not exclude the presence or addition of one or several other features, values, steps or components, or groups thereof. So, the extent of the expression “a device comprising items A and B” should not be restricted to devices consisting of components A and B only. It means that in respect of the present invention, A and B are the only relevant components of the device.

Reference throughout this specification to “one embodiment” or “an embodiment” means that a specific feature, structure or characteristic described in connection with the embodiment has been included in at least one embodiment of the present invention. So, the occurrence of the expressions “in one embodiment” or “in an embodiment” in various places throughout this specification do not necessarily need to refer to the same embodiment all the time, but can do so. Furthermore, the specific features, structures or characteristics may be combined in any suitable manner as would be clear to a person skilled in the art on the basis of this publication, in one or several embodiments.

Similarly, it should be appreciated that in the description of sample embodiments of the invention, various features of the invention are sometimes grouped together in one single embodiment, figure or description thereof intended to streamline the publication and to help the understanding of one or several of the various inventive aspects. This method of publication should therefore not be interpreted as a reflection of an intention that the invention requires more features than explicitly mentioned in each claim. Rather, as the following claims reflect, inventive aspects lie in fewer than all features of one single previously publicised embodiment. So, the claims following on from the detailed description have been explicitly included in this detailed description, with every independent claim being a separate embodiment of the invention.

Furthermore, while some embodiments described herein comprise some, but not other, features included in other embodiments, combinations of features from various embodiments are intended to be within the scope of the invention, and form these various embodiments as would be understood by the person skilled in the art. For example, in the following claims, any of the embodiments described may be used in any combination.

In the description provided here, a large number of specific details are raised. It may therefore be understood that embodiments of the invention may be embodied without these specific details. In other cases, well-known methods, structures and techniques are not shown in detail in order to keep this description clear.

In a first aspect, the present invention relates to a system 100 for supporting a surgical intervention to be carried out on a patient. The system comprises at least one means of identification 120 that can be fitted to the patient, medical instruments 130 provided with means of identification 120, the medical instruments being required for a certain surgical intervention. In addition, the system also comprises an electronic identification system 110 e.g. a computer/iPad or any other tablet or identification system (hereinafter referred to as ‘computer system’). The computer system 110 comprises a list 112 stating the medical instruments 130 required to carry out the surgical intervention on the patient. The computer system has been amended to identify and process the various means of identification 120 in an electronic manner 150. The computer system 110 has furthermore also been amended to execute a computer program 118 that allows the surgical intervention to happen only when the means of identification 120 have been identified which correspond with the medical instruments 130 stated on the list 112. The computer system 110 establishes a link between the parameters entered for preparing the intervention such as (without being restrictive or exhaustive) (1) the patient's identification details, (2) the nature of the intervention planned, (3) the materials anticipated, etc. In embodiments of the present invention, the medical instruments comprise at least a surgical drape, at least one means of identification having been fitted to the surgical drape positioned such that the means of identification can be identified only when the surgical drape has been unfolded. The computer system has thereby been amended to identify the means of identification in an electronic manner and the program has been amended to derive a surgical intervention from at least one means of identification on the surgical drape and to allow a surgical intervention to happen only when the derived surgical intervention corresponds with the surgical intervention to be carried out.

In this manner, a closed circuit is formed between the computer system 110 and the medical instruments 130. Only after verification, by the computer system, of the correctness in the links between the parameters entered as described above and the presence of the medical instruments required for a well-determined surgical intervention, the green light can be given by the computer system for initiating the surgical intervention. In embodiments of the present invention, the medical instruments required are those instruments stated on the list 112 (which corresponds with the specific surgical intervention) of the computer system 110. In some cases in which a medical instrument can be used for several operations but in which another use is required, depending on the operation determined, the means of identification 120 can be fitted such that they are visible or can be identified electronically only in the case of correct use for this specific intervention. The unique and innovative aspect of this invention is that a closed loop is therefore created between, on the one hand, the patient's details/operation in the computer system and, on the other hand, the materials anticipated in the computer system. Finally, also the electronic means of identification, which the patient fitted him or herself in the form of performance scoring lists, can be used to allow an intervention to happen or not. This could prevent that patients who are insufficiently affected, or whose performance is insufficiently poor, would have the intervention. This creates an extra-closed loop, the intervention not being based on a planning only, but also on a performance level of the patient who has also confirmed the intervention within the program.

The result is that the Safety Check happens via at least 3 channels:
(1) Traditional channel: The identification and ticking of the parameters established such as identification patient and intervention (traditional Safety Check),
(2) New channel: The feedback from the materials anticipated happens through the coding applied and becomes possible only when all the parties involved have confirmed the intervention within the computer system first.

The computer system links both channels and thus provides feedback that all the links entered via the ‘traditional channel’ correspond with the links in the ‘new channel’.

In embodiments of the present invention, the surgical intervention 116 to be carried out on the patient is saved in the computer system 100. It is an advantage that it can be prevented as such that an incorrect surgical intervention is carried out. In embodiments of the present invention, lists of the medical instruments required are maintained per surgical intervention. In embodiments of the present invention, the patient's identification information 114 is maintained in the computer system. This can for example be the patient's name.

The computer system 110 can for example be a laptop or a desktop, it can also be a tablet or a smart phone or another identification device. The program 118 can for example be an application. The computer system 110 has been amended to identify the means of identification 120 in an electronic manner. This can for example happen by means of a camera or scanner. The means of identification is a ‘coding’ or a ‘means of coding’ in the most general sense so that a direct communication is made possible between the computer system and the medical materials to which the ‘means of identification’ is fitted. For example, the means of identification can be a bar code or a QR code or another code. Furthermore, the means of identification can also be a sensor that is programmed or ‘tagged’. This can be a sensor or detector that is disposable or reusable, this detector can consist of all sorts of forms or materials. In other embodiments of the present invention, the label can for example be an RFID tag. In this case, the computer system must have been amended to be able to read such RFID tags. The means of identification can also be a label, an identification such as a reference or product number being present, that can be scanned and read using character recognition. Alternatively, any other embodiment that enables an electronic reading of the means of identification can also be applied. The computer system 110 can also be linked to a network. In this case, the program 118 can also be an online application which can furthermore exchange with other systems. As such, patient identification details, surgical interventions, and lists of medical instruments required for example can be exchanged. The computer system and the integration of the feedbacks from the means of identification and the patient details and follow-up modalities allow a unique database of details linked to the closed loop system to be constructed. The code provides a direct communication of the medical material to the computer system so that the computer system can establish a link between the patient details and the efficiency of the material anticipated. It is not only a notification of the ‘nature of the material’ (e.g. an analogue prosthesis number) but particularly a communication of the ‘efficiency of the material for a certain intervention for a specific patient’ (e.g. this prosthesis with this number is anticipated for this patient for this intervention on this side (i.e. a link is established between the traditional channel and the new channel). It can also be a communication with the patient's performance level and rehabilitation schedule.

In embodiments of the present invention, a patient can be scheduled into the computer system for a surgical intervention. Thereby, the patient identification information 114 and the surgical intervention 116 required on the patient are saved in the computer system 110. Also, a list 112 of the various medical instruments required for the surgical intervention in question is saved into the computer system 110. This can be retrieved from a memory based on the type of surgical intervention 116 to be carried out, or can be read in the preparation of the operation.

In embodiments of the present invention, the program 118 has been amended in order to check that the correct intervention is scheduled. Additionally it is also possible to check that it relates to the correct patient. Also, the patient details can for example be identified via a label, for example via a label linked to a patient's identification band. Alternatively, other means of identification can be used here too. Furthermore, the green light for the start of the surgical intervention can be given by the program only when these medical instruments and this patient have been identified corresponding with the medical instruments stated on the list. In embodiments of the present invention, a one-to-one relationship is achieved in this manner between the details the list 112 in the computer system 110 and the medical instruments 130 made available for the surgical intervention. Also, the intervention can be started up only once the patient has created or formulated a unique confirmation of the intervention.

In embodiments of the present invention, during the preparation and at the start of an operation, the various medical instruments 120 identified are identified and linked in an electronic manner to the identification of the patient and the intervention. Based on this identification, the program checks that the medical instruments opened correspond with the surgical intervention anticipated.

The medical instruments 130 can for example be operation instruments 132, instruments such as disinfectants etc. or other attributes, but in present embodiments, always comprise a surgical cloth 134 for covering the surgical area in a sterile manner. Also, the medical instruments can also be prosthesis materials or other materials which are to be scheduled or applied to the patient either temporarily or permanently and are even saved in the performance questionnaires to the performance level of the patient as this one. A label/sensor/other means of identification etc. is fitted to the surgical drape 134 which, after their identification, allows a surgical intervention to be determined associated with said label. The label/sensor can for example comprise a unique code for this. Alternatively, the other means of identification such as described above can be used too. If said surgical drape 134 can be used for various interventions, the means of identification can be positioned such that it becomes visible only when the surgical drape 134 is fitted in the correct manner for the specifically intended intervention. The ultimate step is the feedback from the final identification system that is fitted to the drape and is activated only as the last component of the last steps of the preparatory process. By feedback from this identification system, the various aforementioned parameters are cross-checked and the ‘closed loop’ is created. This final means of identification can be activated in all sorts of different manners, such as an electronic activation, an exposing of the means and so on.

In embodiments of the present invention, the surgical drape 134 and/or the other medical materials comprise a means of identification 120 from which, after their identification, a surgical intervention linked to a specific patient can be derived. In this manner, the program can compare the surgical intervention indicated by the label with the surgical intervention to be carried out and confirm that it relates to the surgical intervention 116 to be carried out. The intervention can be checked only if all the links during the preparatory process perfectly match the identification of the patient, the intervention and the materials and actions (whether surgical, anaesthesiology or any other medical act) anticipated. Also, the patient's performance level, based on the performance questionnaires within the computer system, can be used as a parameter too for allowing the intervention to happen or not.

It is an advantage of embodiments of the present invention that the details in the computer system (e.g. patient identification information 114, surgical intervention 116, medical instruments list 112) can be unambiguously linked to the reality in the operating room (the patient, the surgical intervention, and the medical instruments). The patient can for example be identified in an electronic manner using a label 120 on the patient, the surgical intervention 116 can for example be identified in an electronic manner by input by the physician, the medical instruments 130 such as a surgical drape can for example be identified by a means of identification 120 on the medical instruments 130. In this manner, a one-to-one relationship is achieved between the data in the computer system and the reality in the operating room (both becoming one and indivisible). Therefore, a ‘closed loop’ is achieved between the ‘traditional channel’ (the traditional Safety List) and the ‘new channel’ (the feedback from the material based on the coding fitted). In this manner, a closed ‘loop’ is formed so that the risk of an incorrect surgical intervention is at least minimised.

In embodiments of the present invention, the surgical drape 134 is made such that, for fitting it to the patient, a label/sensor from another identification system 120 is present that allows to identify that the surgical drape is suitable for one or several specific surgical interventions. This identification system can then for example be identified by the computer system. The system can have a unique code that is associated with a certain intervention. The system can be a label or code fitted as such in advance, but can also be a sensor that is coded. In embodiments of the present invention, the program 118 has been amended to verify that the surgical intervention associated with the label 120 corresponds with the surgical intervention 116 to be carried out. A similar system can be used for all other medical materials such as prostheses, instruments to carry out operations, etc.

In embodiments of the present invention, the label 120 on the surgical drape 134 is released or made visible or accessible for the computer system during the preparation of the operation and this in the site where the physician or the team believe the operation or other act needs to be done. Also, it can be a signal too that is sent from a sensor or a detector to the computer system. By identifying this label 120 electronically, a surgical intervention 116 is achieved that is associated with this label. This can then be compared against the surgical intervention to be carried out. When the two differ, the system will not allow the surgical intervention to happen or a warning will appear indicating a conflict in the links between the various ‘channels’ described above.

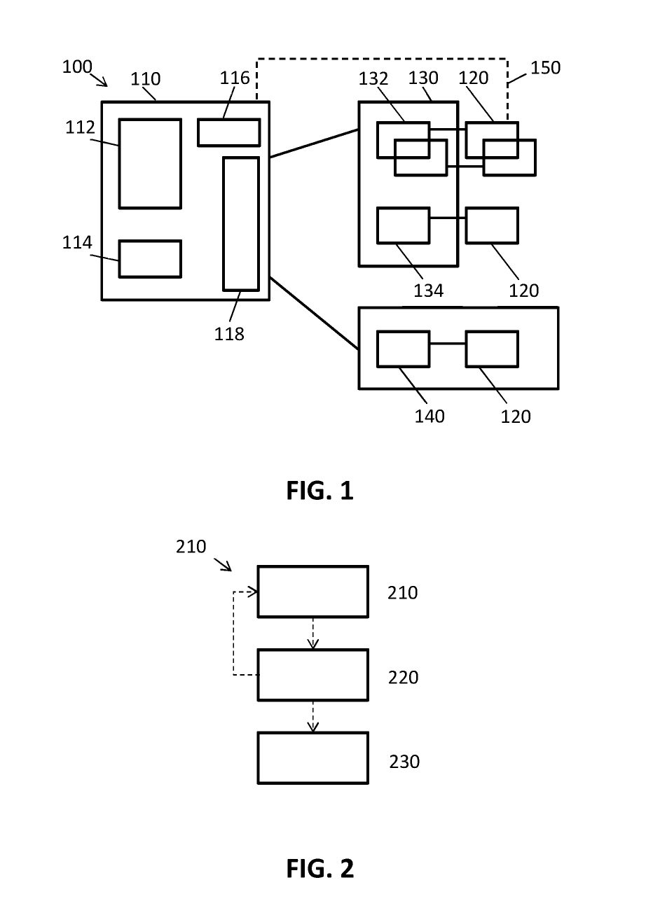

FIG. 1 is a schematic representation of a system 100 for supporting a surgical intervention on a patient. The system 100 comprises various means of identification 120, here for example labels, that can be fitted to medical instruments 130. The medical instruments 130 can for example be operation instruments 132 or one or several surgical drapes 134. The figure also shows a label 120 on a patient 140. This label can be used to identify the patient in an electronic manner. The system 100 also comprises a computer system 110. The computer system 110 comprises, among other items (without being restrictive), (1) the identification details of the patient and the intervention scheduled and (2) a list 112 of the various medical instruments required for the surgical intervention. The computer system has been amended to identify the labels 120 in an electronic manner. In this figure, this is illustrated using the dotted lines 150. The computer system has been amended to execute a program 118 that allows the surgical intervention to happen only when these labels have been identified which correspond with the medical instruments 130 stated on the list 112. In this example, the computer system comprises patient identification information 114 and the surgical intervention 116.

In some embodiments, the computer program has furthermore also been programmed to register a patient result before and after the intervention. In this manner, both a result for the intervention and the patient safety procedure can be registered collectively. Also, even an operation can be made possible only if the patient has a certain performance or pain level. The computer program also allows the patient to be assisted in their performance and rehabilitation, to be monitored and a database of these details to be constructed.

In a second aspect, the present invention relates to a method 200 for supporting a surgical intervention to be carried out. This method comprises at least two steps. An identification step 210 during which various medical instruments are identified in an electronic manner. For this, means of identification are fitted to the various medical instruments. The medical instruments comprise at least a surgical drape, at least one means of identification having been fitted to the surgical drape positioned such that the means of identification can be identified only when the surgical drape has been unfolded. During a verification step 220, a list of medical instruments required for the surgical intervention is tested using the medical instruments identified. A surgical intervention is derived from at least one means of identification on the surgical drape. The operation is allowed to happen only when these labels have been identified which correspond with the medical instruments stated on the list and when the derived surgical intervention corresponds with the surgical intervention to be carried out.

FIG. 2 shows a possible succession of the various steps. The various steps in this figure are the identification step 210, the verification step 220 and the confirmation step 230. The identification step 210 and the verification step 220 can thereby be carried out alternately. Both can be split up into smaller components that can follow one another. In embodiments of the present invention, the method comprises a confirmation step 230 in which the surgical intervention to be carried out is confirmed by a person. This can for example be the patient, a physician, or a logistics worker.

In embodiments of the present invention, the patient is identified during the identification step 210, for example using a label on the patient. This identification can be used to log the patient in on the computer system. This identification can also be used to verify that a patient already logged in on the computer system has been logged in correctly.

The order of the steps in FIG. 2 is by way of example. The invention is not restricted to this order.

An example embodiment of the present invention is discussed in the following paragraphs. This concerns a method in which, in a first step, the physician enters the name of the patient, the date of birth, the operation and operation date into the computer system.

E.g. KC, 8/11/1977, THA re (total hip arthroplasty right) on 31/08/2016

In a next step, the patient signs their agreement for the intervention electronically. As such, the patient gives a confirmation for the surgical intervention. Also, the patient can monitor their performance using performance questionnaires.

E.g. patient confirmation, KC, 8/11/1977, THA re on 31/08/2016

In a next step, the person in charge of logistics makes the instrument kit (the medical instruments) available for the THA re intervention. This kit contains instruments that are labelled as instruments that can be used for a THA re only. E.g. It contains a surgical drape marked for a THA re. When preparing the medical instruments, the medical instruments are identified by the computer system in an electronic manner. The person in charge of logistics can for example take photos of the labels on the medical instruments. Based on the label information, the program in the computer system can check that the labels have been identified which correspond with the medical instruments stated on the list.

E.g. verification list OK, KC, 8/11/1977, THA re on 31/08/2016

In a next step, the anaesthetist can also confirm that the surgical intervention indicated by the computer system is the correct surgical intervention. Also, the anaesthesia can also be carried out after confirmation within the computer system. If for example the performance level of the patient, as indicated within the computer program, is not sufficient, then it can even be that the anaesthesia is not allowed by the program.

E.g. anaesthetist confirmation, KC, 8/11/1977, THA re on 31/08/2016

In a next step, the patient confirms the surgical intervention to be carried out for a second time.

E.g. patient reconfirmation, KC, 8/11/1977, THA re on 31/08/2016

In a next step, e.g. at the end of the preparatory process, the label or the sensor on the surgical drape must be released before initiating the intervention. This label is identified by the computer system or the sensor communicates with the computer system. The information of the label or the sensor corresponds with the surgical intervention anticipated on the patient in question. The program verifies that this intervention corresponds with the surgical intervention to be carried out.

E.g. When pulling the foil off the drape, a label is released. This is photographed/captured and the application/computer system identifies the label as the one of KC, 8/11/1977, THA re on 31/08/2016. In embodiments of the present invention, the label indicates a surgical intervention only. Also, the operation can also be carried out after confirmation within the computer system. If for example the patient's performance level, as indicated within the computer program, is not sufficient, then it can even be that the operation is not allowed by the program. This unique link with the patient's performance prevents that patients who are still too ‘healthy’ or ‘perform too well’ would be operated.

Other medical instruments too can be identified in this manner.

After carrying out these steps, the program confirms that the operation can or cannot be carried out safely. As such, the program has established the link between the information of the ‘traditional channel’ (Safety check list) on the one hand and the new channel (material communication from label or sensor or performance lists).

If, due to an error, the surgical drape is opened at an incorrect site, for example for an operation to the left hip (for example on the left side), then another label is released in this site (e.g. THA li). Since the surgical intervention indicated by this label does not correspond with the surgical intervention to be carried out, the program will indicate that the intervention cannot be carried out.

In a third aspect, the present invention relates to a medical instrument to which a label has been fitted so that it can be identified by a system corresponding with embodiments of the present invention. The medical instrument is a surgical drape that comprises a means of identification that can be identified only after the surgical drape has been unfolded.

The various aspects can easily be combined, and the combinations will also correspond with embodiments according to the present invention.

1.-12. (canceled)

13. A system for supporting a surgical intervention to be carried out on a patient, the system comprising:
a means of identification that can be fitted to the patient, medical instruments with means of identification for a surgical intervention, and
a computer system,
the computer system comprising a list of the various medical instruments required for the surgical intervention to be carried out, the computer system having been amended in order to identify the medical instruments using the means of identification in an electronic or other manner, and the computer system having been programmed to execute a program that will allow the surgical intervention to happen only if these means of identification have been identified which correspond with the medical instruments stated on the list, and in which pre and post operation performance lists can be used and maintained,
the medical instruments comprising at least a surgical drape, at least one means of identification having been fitted to the surgical drape positioned such that the means of identification can be identified only if the surgical drape has been unfolded, the computer system being programmed to identify the means of identification in an electronic manner and the program being programmed to derive a surgical intervention from at least one means of identification on the surgical drape and allowing a surgical intervention to happen only if the derived surgical intervention corresponds with the surgical intervention to be carried out.

14. The system according to claim 13, the program furthermore requiring that at least one confirmation about the surgical intervention to be carried out is given by a person before the program allows the operation to happen.

15. The system according to claim 13, the program furthermore requiring that a confirmation about the surgical intervention to be carried out is given by the patient, the person in charge of logistics and the medical and/or paramedical team before the system allows the operation to happen.

16. The system according to claim 13, the system comprising a label that can be fitted to the patient, this label allowing the patient to be identified.

17. The system according to claim 13, the at least one means of identification having been positioned on the surgical drape such that if the surgical drape is positioned on the patient correctly, the identification of the means of identification corresponds with the site of a surgical intervention that must happen at the site of the means of identification.

18. The system according to claim 13, the surgical drape comprising protection foils and the means of identification being released only after one or several protection foils have been removed or the means of identification gives the signal and communicates with the computer program only when the medical team activates the means of identification.

19. The system according to claim 13, the computer program furthermore being programmed to register a patient result before and after the intervention.

20. A method for supporting a surgical intervention to be carried out, the method comprising:
an identification step for identifying the patient and the intervention and one or several medical instruments in an electronic manner using a means of identification on the medical instruments, the medical instruments comprising at least a surgical drape, at least one means of identification having been fitted to the surgical drape and positioned such that the means of identification can be identified only when the surgical drape has been unfolded,
a verification step for checking a list of medical instruments required for the surgical intervention using the identified medical instruments, a surgical intervention being derived from the at least one means of identification on the surgical drape and the operation being allowed only when these means of identification have been identified which correspond with the medical instruments stated on the list and when the derived surgical intervention corresponds with the surgical intervention to be carried out.

21. The method according to claim 20, the method furthermore comprising a confirmation step in which the surgical intervention is confirmed by a person.

22. The method according to claim 20, in which the patient too is identified during the identification step using a label on the patient.

23. The method according to claim 20, in which before or during the verification step, protection foils are removed from the surgical drape so that the label is released or the sensor is activated.

24. A medical instrument to which a label has been fitted that can be identified by a system according to claim 13, in which the medical instrument is a surgical drape that comprises a means of identification that can be identified only after the surgical drape has been unfolded.
